(19) United States
(12) Patent Application Publication
(10) Pub. No.: US 2023/0273146 A1
(43) Pub. Date: Aug. 31, 2023

(54) SAMPLE HANDLING FOR DIAGNOSTICS

(71) Applicant: QSM Diagnostics, Inc., Boston, MA (US)

(72) Inventors: Edgar D. GOLUCH, Somerville, MA (US); Nicholas CADIROV, Somerville, MA (US)

(21) Appl. No.: 18/017,927

(22) PCT Filed: Jul. 28, 2021
(86) PCT No.: PCT/US21/43386

§ 371 Date: Jan. 25, 2023

Related U.S. Application Data
(60) Provisional application No. 63/057,665, filed on Jul. 28, 2020.

(51) Int. Cl. G01N 27/327; C12Q 1/04; G01N 1/34; G01N 27/48

(52) U.S. Cl. CPC G01N 27/3277; C12Q 1/04; G01N 1/34; G01N 27/48; A61B 2010/0054

(57) ABSTRACT

The present invention relates to, inter alia, methods and kits for detection of microbes. More particularly methods and kits for detecting an infection in a subject's ear suitable for sample types including ear wax are provided. The method comprises measuring a presence, absence or amount of a compound in the ear wax by electrochemically measuring a redox active compound using an electrochemical sensor.

### PRIORITY

This application claims the benefit of, and priority to, U.S. Provisional Application No. 63/057,665, filed Jul. 28, 2020; the contents of which are herein incorporated by reference in their entirety.

### TECHNICAL FIELD

The present invention relates to, inter alia, methods and kits for detection of microbes.

### BACKGROUND

Point-of-care (POC) tests are performed at or near the site where a patient initially encounters the health care system, have rapid turnaround times, and provide actionable information that can lead to a change in health management. Rapid results reduce the need for follow-up visits and enable timely administration of specific treatment, rather than the reliance on the treatment of a suspected/presumptive diagnosis based on solely symptoms, existence of risk factors, etc. For example, physicians or veterinarians may prescribe antibiotics for suspected bacterial infections, but POC diagnostics facilitate antibiotic stewardship by quickly providing specific diagnosis. Rapid diagnostic tests work by detecting analytes such as microbial antigens and patient antibodies that are found in clinical samples.

Most POC rapid diagnostics use the lateral flow (LF) platform and usually require an optical or colorimetric readout. These tests are suitable for samples such as blood and urine. However, many common infections and disease require diagnosis in other sample types, which cannot run on these common diagnostic tests due to their opaqueness or flow limitations. Accordingly, there remains a need for novel methods of diagnosis for these kinds of samples.

### SUMMARY

Accordingly, the present disclosure provides, in part, methods and kits for detecting an infection in a subject's ear suitable for sample types including cerumen (ear wax). In some aspects, the present disclosure provides a method for detecting an infection in a subject's ear. In some embodiments, the method comprises: (a) obtaining a sample of cerumen by adsorbing the cerumen onto an applicator, (b) extracting the cerumen from the applicator, and (c) measuring a presence, absence or amount of a compound in the cerumen, wherein the compound may be redox-active and associated with the infection. In this aspect, the measuring comprises contacting the compound with an electrochemical sensor comprising a working electrode and a reference electrode, and electrochemically measuring a current flow. In some embodiments, the current flow is correlated with the presence, absence or amount of the compound. In some embodiments, the extraction of the cerumen from the applicator comprises contacting the applicator with a solvent.

In some embodiments, the extracting removes a substantial amount of the cerumen and/or the compound from the applicator. In some embodiments, the extracting occurs in a collection tube, on the surface of the electrochemical sensor, and/or on a hydrophilic membrane attached to or contacted with the electrochemical sensor. In some embodiments, the hydrophilic membrane wicks the cerumen from the applicator.

In some embodiments, the applicator used is a sterile swab, optionally having the adsorbent portion of substantially cotton, substantially foam, substantially calcium alginate, substantially nylon, substantially polyester, substantially polyethylene, substantially flocked polyester, or substantially rayon, or is a sterile curette. In some embodiments, the subject is a non-human animal, such as a captive animal, a pet animal, a farm animal, or a zoo animal. In some embodiments, the subject is a canine or a feline. In some embodiments, the subject is a dog.

In some embodiments, the infection is a Pseudomonas aeruginosa infection, optionally selected from one or more of otitis externa, otitis media, and otitis interna. In some embodiments, the compound, which may be a redox-active compound associated with the infection, is a quorum sensing molecule. In some embodiments, the quorum sensing molecule is a phenazine compound. In some embodiments, the phenazine compound is pyocyanin. In some embodiments, the presence of pyocyanin is indicative of the presence or extent of Pseudomonas aeruginosa infection.

In some embodiments, the solvent used for extracting the cerumen from the applicator is an aqueous solution. In some embodiments, the solvent is saline. In some embodiments, the solvent comprises ethanol or an aqueous solution thereof. In some embodiments, the solvent comprises about 1%, or about 2.5%, or about 5%, or about 7.5%, or about 10%, or about 12.5%, or about 15%, or about 17.5%, or about 20%, or about 25% ethanol.

In embodiments, the solvent comprises a phosphate buffered saline (PBS) and an alcohol. In embodiments, the alcohol is ethanol. In embodiments, the solvent comprises PBS comprising ethanol in an amount of about 0.1% to about 25%, or about 0.2% to about 20%, or about 0.5% to about 15%, or about 1% to about 10%, or about 2% to about 8%, or about 2.5% to about 7.5%, or about 3% to about 7%, or about 4% to about 6%. In embodiments, the solvent further comprises about 1 mM MgCl₂. In embodiments, the solvent comprises PBS comprising about 5% ethanol. In embodiments, the solvent comprises about 0.1 mM to about 5 mM, or about 0.25 mM to about 3 mM, or about 0.5 mM to about 2 mM, or about 0.75 mM to about 1.25 mM MgCl₂ or MgSO₄.

In some embodiments, the electrochemical measurement is selected from square wave voltammetry, linear sweep voltammetry, staircase voltammetry, cyclic voltammetry, normal pulse voltammetry, differential pulse voltammetry, and chronoamperometry. In some embodiments, the electrochemical measurement is square wave voltammetry and the current flow is measured in response to one or more square wave potentials.

In some embodiments, the working electrode is comprised of gold (Au), silver (Ag), platinum (Pt), indium tin oxide (ITO), carbon, carbon nanotubes, carbon nanofibers, graphene, carbon-platinum composites, carbon nanotubes with gold nanoparticles, and any combination thereof.

In some embodiments, the reference electrode is comprised of silver (Ag), silver chloride (AgCl), gold (Au), palladium (Pd) and platinum (Pt), and any combination thereof.

In some embodiments, the method informs the administration of one or more antibiotics upon a positive test for infection, the withholding of one or more antibiotics upon a negative test for infection and/or the selection of an appropriate antibiotic for the infectious agent upon a positive test for infection.

In some aspects, the present disclosure provides a kit for detecting an infection in a subject's ear. The kit comprises (a) an applicator suitable for adsorbing a sample of cerumen; (b) a solvent suitable for extracting the cerumen and/or a compound within the cerumen from the applicator; and (c) an electrochemical sensor, the electrochemical sensor comprising a working electrode, a counter electrode and a reference electrode and being suitable for electrochemically measuring a current flow through the sensor, which is correlated with the presence, absence or amount of the compound.

In some aspects, the present disclosure provides a kit for detecting an infection in a subject's ear. The kit comprises (a) an applicator suitable for adsorbing a sample of cerumen; (b) a solvent suitable for extracting the cerumen and/or a compound within the cerumen from the applicator; and (c) an electrochemical sensor, the electrochemical sensor comprising a working electrode and a reference electrode and being suitable for electrochemically measuring a current flow through the sensor, which is correlated with the presence, absence or amount of the compound.

In some embodiments, the electrochemical sensor further comprises a counter electrode. In some embodiments, the counter electrode of each sensor is identical to the working electrode.

In some embodiments, the kit further comprises one or more of positive control samples, negative control samples, a key for estimating a number of viable cells of a microorganism, and instructions to use. In some embodiments, the kit further comprises a collection tube. In some embodiments, the positive control comprises Pseudomonas aeruginosa cells or a metabolite thereof. In some embodiments, the positive control comprises pyocyanin.

In some embodiments, the electrochemical sensor generates a waveform suitable for performing an electrochemical measurement selected from square wave voltammetry, linear sweep voltammetry, staircase voltammetry, cyclic voltammetry, normal pulse voltammetry, differential pulse voltammetry, and chronoamperometry. In some embodiments, the electrochemical sensor is capable of performing square wave voltammetry, wherein the current flow is measured in response to one or more square wave potentials.

In some embodiments, the electrochemical sensor comprises a second working electrode and the working electrode is one of an oxidizing electrode and a reducing electrode, and the second working electrode is the other of the oxidizing electrode and the reducing electrode. In some embodiments, the working electrode is comprised of gold (Au), silver (Ag), platinum (Pt), indium tin oxide (ITO), carbon, carbon nanotubes, carbon nanofibers, graphene, carbon-platinum composites, carbon nanotubes with gold nanoparticles, and any combination thereof. In some embodiments, the reference electrode is comprised of silver (Ag), silver chloride (AgCl), gold (Au), palladium (Pd), and platinum (Pt), and any combination thereof.

Any aspect or embodiment described herein can be combined with any other aspect or embodiment as disclosed herein.

### BRIEF DESCRIPTION OF THE DRAWINGS

FIG. 1 shows a swab sample containing exudate and wax obtained from a dog's ear.

FIG. 2 shows an example of an electrochemical scan with a peak current located at −0.29 V. 100 μL of redox molecule spiked saline solution was pipetted onto the sensor to fully wet all the electrodes simultaneously.

FIG. 3 shows an illustrative electrochemical sensor with the three types of electrodes (working, counter, and reference electrodes) as labeled.

FIG. 4 shows illustrative sample collection. A swab was placed in 50 μL of saline solution. The swab immediately absorbed all of the saline.

FIG. 5 shows a foam-tipped swab (upper swab) compared to a cotton-tipped swab (lower swab).

FIG. 6 shows a foam-tipped swab placed in 100 μL of saline solution.

FIG. 7 shows an illustrative electrochemical sensor with a 300 mesh nylon membrane covering the exposed working, counter, and reference electrodes.

FIG. 8 shows a comparison of electrochemical scans of spiked animal cerumen extracted with either a saline solution alone or a saline solution comprising an alcohol additive.

FIG. 9 shows a square-wave voltammetry scans of pyocyanin (PYO) in different concentrations of ethanol added to phosphate buffered saline solution (PBS).

### DETAILED DESCRIPTION

The present disclosure is based, in part, on the discovery that the exudate material from an ear swab may be transferred to specialized electrochemical sensors disclosed herein, optionally using specialized applicators disclosed herein for diagnostic purposes. Accordingly, the present disclosure provides, in part, methods and kits for detecting an infection in a subject's ear suitable for sample types including cerumen (ear wax). Surprisingly, the methods disclosed herein allow efficient extraction of cerumen sample and require lesser amount of cerumen fluid extract compared to the known methods.

In some aspects, the present disclosure provides a method for detecting an infection in a subject's ear, comprising, (a) obtaining a sample of cerumen, (b) extracting the cerumen from the applicator, and measuring a presence, absence or amount of a compound in the cerumen, wherein the compound may redox-active and associated with the infection. In some embodiments, the measuring comprises contacting the compound with a microfluidic sensor comprising a working electrode and a reference electrode, and electrochemically measuring a current flow. In some embodiments, the microfluidic sensor comprises a working electrode, a reference electrode, and a hydrophilic membrane that helps transfer the compound from the applicator to the entire electrochemical sensor surface. In some embodiments, the measuring comprises contacting the compound with an electrochemical sensor comprising a working electrode and a reference electrode, and electrochemically measuring a current flow. In some embodiments, the current flow is correlated with the presence, absence or amount of the compound. In some embodiments, the cerumen is adsorbed onto an applicator. In some embodiments, the extraction comprises contacting the applicator with a solvent.

In various embodiments, the present disclosure provides methods and kits for veterinary use, e.g. for the diagnosis of ear infections in a dog.

Electrochemical Sensors

In some aspects, the present disclosure provides a method for detecting an infection in a subject's ear comprising, measuring a presence, absence or amount of a compound in the cerumen by contacting the compound with an electrochemical sensor, and electrochemically measuring a current flow. In this aspect, the electrochemical sensor comprises a working electrode and a reference electrode.

In some embodiments, the electrochemical sensor further comprises a hydrophilic membrane attached to, contacted with or adjoined to the working electrode and/or the reference electrode.

In some embodiments, the working electrode material of one or more sensors is selected from gold (Au), silver (Ag), platinum (Pt), indium tin oxide (ITO), carbon, multi-walled carbon nanotubes, single-walled carbon nanotubes, carbon nanofibers, graphene, carbon-platinum composites, multi-walled carbon nanotubes with gold nanoparticles, and any combination thereof. In some embodiments, the working electrode has a diameter between about 0.1 mm and about 10 mm, optionally between about 1 mm and about 5 mm. In some embodiments, the working electrode has a diameter between about 1.5 mm and about 4 mm.

In an illustrative embodiment, the working electrode comprises about 1.5 mm gold screen-printed at elevated temperature. In another illustrative embodiment, the working electrode comprises about 1.5 mm platinum. In another illustrative embodiment, the working electrode has electrodeposition of gold to coat copper electrodes exposed on a printed circuit board. In another illustrative embodiment, the working electrode has screen-printed carbon paste to coat copper electrodes exposed on a printed circuit board. In another illustrative embodiment, the working electrode comprises about 4 mm gold. In yet another illustrative embodiment, the working electrode comprises about 1.5 mm Au screen-printed at low temperature. In yet another illustrative embodiment, the device may comprise an oxidizing and a reducing working electrode, for amplifying the signal, and the two working electrodes consist of gold and or platinum.

In some embodiments, the working electrodes may make up a wall or part of a wall of a channel, such as a microfluidic channel or a nanofluidic channel, into which the fluid sample is introduced and within which the redox reaction takes place. In some embodiments, the oxidizing electrode and the reducing electrode are separated by a distance of about 20 nm to 1 mm or greater. In some embodiments, the distance between the oxidizing electrode and the reducing electrode is from 20 nm to about 100 nm, or from about 20 nm to about 40 nm, or from about 40 nm to about 60 nm, or from about 60 nm to about 80 nm, or from about 80 nm to about 100 nm, or from about 100 nm to about 150 nm, or from or from about 50 nm to about 500 nm, or from about 100 nm to about 1 μm, or from about 500 nm to about 5 μm, or from about 1 μm to about 10 μm, or from about 5 μm to about 50 μm, or from about 10 μm to about 100 μm, or from about 50 μm to about 500 μm, or from about 100 μm to about 1 mm, or greater. In some embodiments, the distance between the oxidizing electrode and the reducing electrode is from 20 nm to about 100 nm, or from about 20 nm to about 40 nm, or from about 40 nm to about 60 nm, or from about 60 nm to about 80 nm, or from about 80 nm to about 100 nm, or from about 100 nm to about 150 nm.

The surface area of the working electrodes can be selected to accommodate a desired size of the device. Without being bound by theory, larger surface area generally improves the signal and sensitivity of the device. For example, in different embodiments, the surface area of each working electrode can be about 100, about 200, about 300, about 400, about 500, about 800, about 1000, about 2000, about 3000, about 5000, about 10000, about 50000, about 100000, about 200000, or about 500000 nm², or about 1, about 2, about 5, about 10, about 50, about 100, about 200, about 300, about 400, about 500, about 800, about 1000, about 2000, about 3000, about 5000, about 10000, about 50000, about 100000, about 200000, or about 500000 μm², or about 1, about 2, about 4, about 7 mm² or greater. In different embodiments, the surface area of each working electrode can be about 100, about 200, about 300, about 400, about 500, about 800, about 1000, about 2000, about 3000, about 5000, about 10000, about 50000, about 100000, about 200000, or about 500000 nm², or about 1, about 2, about 5, about 10 μm², or greater.

Any reference electrode that is compatible with the chosen working electrode may be used. In some embodiments, the reference electrode material of one or more sensors is selected from silver (Ag), silver chloride (AgCl), and platinum (Pt). In some embodiments, the reference electrode comprises silver (Ag). In some embodiments, the reference electrode comprises Ag/AgCl.

In some embodiments, the electrochemical sensor further comprises a counter electrode. In some embodiments, the counter electrode of each sensor is identical to the working electrode.

In some embodiments, the electrochemical sensor may be used for measuring an electrochemical reaction taking place at the working electrode at a well-defined potential. In some embodiments, the electrochemical reaction taking place at the working electrode is measured in comparison to the electrochemical reaction taking place at the reference electrode. The electrochemical sensor thereby facilitates an electrochemical detection of a predetermined redox-active compound associated with the infection (e.g., without limitation, pyocyanin). In some embodiments, the concentration of a redox-active compound associated with the infection (e.g., without limitation, pyocyanin) is measured by introducing a fluid sample extracted from an applicator into an electrochemical sensor including a working electrode and a reference electrode; performing an electrochemical measurement to detect a redox-active compound associated with the infection (e.g., without limitation, pyocyanin) in the fluid sample extracted from an applicator determining a concentration of the redox-active compound associated with the infection (e.g., without limitation, pyocyanin) in the fluid sample extracted from an applicator by using a previously determined correlation between known concentrations of the redox-active compound associated with the infection (e.g., without limitation, pyocyanin) and a current flow through the working electrode. In some embodiments, the defined potential of the working electrode may be varied, and the response from the electrochemical reaction is seen from the current of the working electrode.

In some embodiments, the electrochemical sensor comprises a second working electrode. In some embodiments, the second working electrodes with respect to one or more of surface area, size, material, and coating. In some embodiments, the electrochemical sensor may include an oxidizing working electrode and a reducing working electrode. In some embodiments, the concentration of a redox-active compound associated with the infection (e.g., without limitation, pyocyanin) is measured as current flow through the oxidizing electrode and the reducing electrode. In some embodiments, the working electrode is one of an oxidizing electrode and a reducing electrode, and the second working electrode is the other of the oxidizing electrode and the reducing electrode. A potential suitable for oxidizing the redox-active compound associated with the infection (e.g., without limitation, pyocyanin) is applied at the oxidizing electrode and a potential suitable for reducing the redox-active compound associated with the infection (e.g., without limitation, pyocyanin) is applied at the reducing electrode.

In some embodiments, a given redox-active compound associated with the infection (e.g., without limitation, pyocyanin) electrochemically reacts differently on different electrode surfaces. Thus, different electrode materials and geometries used for chemical detection will give different results. Accordingly, in some embodiments, the sensor array increases the sensitivity and specificity of the measurement, and reduces the noise from other substances present in a biological sample. In some embodiments, the sensor array may comprise two or more sensors, wherein each sensor comprises a working electrode that differs from the other working electrodes with respect to at least one of the following characteristics: surface area, size, material, and coating.

The electrochemical measurement can be made in any suitable manner. In some embodiments, the electrochemical measurement may made by squarewave voltammetry, linear sweep voltammetry, staircase voltammetry, cyclic voltammetry, normal pulse voltammetry, differential pulse voltammetry, and chronoamperometry. In some embodiments, the electrochemical measurement is square wave voltammetry and the current flow is measured in response to one or more square wave potentials. In some embodiments, optionally cyclic voltammetry is used and the working electrode potential is ramped linearly versus time. In some embodiments, the potential is ramped linearly up, and when a set potential is reached, the potential is ramped in the opposite direction to the initial potential, and the cycle is repeated. In some embodiments, the working electrode potential include linear sweep voltammetry, staircase voltammetry, square-wave voltammetry, and differential pulse voltammetry.

In some embodiments, the presence, absence or amount of the compound is measured as current flow through the working electrode. In some embodiments, the presence, absence or amount of compound is measured as current flow through the oxidizing electrode and the reducing electrode.

In some embodiments, the fluid sample extracted from an applicator can be introduced into a well, chamber, or another form of receptacle in which the reaction can take place. The volume of the channel, well, chamber or other receptacle can be less than about 50 nanoliters (nL), less than about 10 nL, less than about 1 nL, less than about 100 picoliters (pL), less than about 50 pL, less than about 10 pL, less than about 5 pL, or less than about 1 pL.

In some embodiments, a sample volume that is introduced into the electrochemical sensor can be less than about 100 μL, less than about 50 μL, less than about 20 μL, less than about 10 μL, less than about 5 μL, less than about 2 μL, or less than about 1 μL.

In some embodiments, a capillary or wicking material can be disposed at or near an inlet of the electrochemical sensor to draw the fluid sample into the device. Is some embodiments, a matrix material can be disposed at or near an inlet of an electrochemical sensor, for example, to isolate the electrodes from the bacteria while permitting passage of pyocyanin to access the electrodes.

In some embodiments, the electrochemical sensor is a microfluidic sensor comprising a working electrode, a counter electrode and a reference electrode. In some embodiments, the microfluidic sensor comprises a working electrode, a counter electrode, a reference electrode, and a hydrophilic membrane that helps transfer the compound from the applicator to the entire electrochemical sensor surface. In these embodiments, the current flows the current flows through the working electrode and the counter electrode. In some embodiments, the counter electrode functions as a cathode and the working electrode is operating as an anode. In alternative embodiments, the counter electrode functions as an anode and the working electrode is operating as a cathode. In some embodiments, the counter electrode has a surface area much larger than that of the working electrode.

In some embodiments, the electrochemical sensor is a microfluidic sensor comprising a working electrode and a reference electrode. In some embodiments, the microfluidic sensor comprises a working electrode, a reference electrode, and a hydrophilic membrane that helps transfer the compound from the applicator to the entire electrochemical sensor surface. In these embodiments, the current flows the current flows through the working electrode and the reference electrode. In some embodiments, the reference electrode functions as a cathode and the working electrode is operating as an anode. In alternative embodiments, the reference electrode functions as an anode and the working electrode is operating as a cathode.

Quorum Sensing Molecules

Quorum sensing is the regulation of gene expression in response to fluctuations in cell-population density. Bacteria use quorum sensing to regulate certain phenotype expressions, which in turn, coordinate their behaviors. Some common phenotypes include biofilm formation, virulence factor expression, and motility. Quorum sensing bacteria produce and release chemical signal molecules called referred herein as quorum sensing molecules that increase in concentration as a function of cell density. Accordingly, detection of quorum sensing molecules is important for determination of presence of virulent bacteria.

If the amount of pyocyanin exceeds a certain level, the bacteria determine that the bacterial population is big enough to initiate virulence. Thus, in some embodiments, the detection of quorum sensing molecules can provide information about the type of bacterial infection and about its state of progression. For example, the amount of a quorum sensing molecule (e.g., without limitation, pyocyanin) in a sample (e.g. cerumen) is an indication of an infection caused by specific bacteria (e.g. Pseudomonas aeruginosa), as well as an indication of the risk of an infectious attack in a chronically infected individual. Thus, monitoring and detection of the level of pyocyanin before the bacterial population has reached the critical level, is important for avoiding serious infections, as well as critical infectious attacks. This may be of particular relevance for subjects with high risk of infections.

In some embodiments, monitoring of quorum sensing molecules (e.g., without limitation, pyocyanin) level may reduce the amount of antibiotics used due to earlier and more precise diagnosis, which will improve the efficiency of a treatment. Thus, in some embodiments, monitoring of selected quorum sensing molecules is used for determining and/or refining antibiotic treatments, and further improve the life quality of the subjects.

Pseudomonas aeruginosa and Ear Infections

Pseudomonas aeruginosa is a Gram-negative and ubiquitous environmental bacterium. It is an opportunistic pathogen capable of causing a wide array of life-threatening acute and chronic infections, particularly in subjects with compromised immune defense. It has been of particular importance since it is the main cause of morbidity and mortality in cystic fibrosis (CF) patients and one of the leading nosocomial pathogens affecting hospitalized patients while being intrinsically resistant to a wide range of antibiotics. Pseudomonas aeruginosa is the most common pathogen isolated from patients who have been hospitalized longer than 1 week, and it is a frequent cause of nosocomial infections. Some Pseudomonal infections are complicated and can be life-threatening. Pseudomonas aeruginosa causes infections of many parts of the body, including wound infections, burn infections, pneumonia, bacteremia, eye infection, ear infection, etc. Ear infections caused by Pseudomonas aeruginosa include otitis externa, otitis media, and otitis interna.

Pseudomonas aeruginosa is one of the most common causative agent of otitis externa, which is also called swimmer's ear. It involves diffuse inflammation of the external ear canal that may extend distally to the pinna and proximally to the tympanic membrane. It is typically a mild external infection that can occur in otherwise healthy subjects. Water containing the bacteria can enter the ear during swimming. Swimmer's ear causes itching, pain, and sometimes a discharge from the ear. The acute form has an annual incidence of approximately 1 percent and a lifetime prevalence of 10 percent. On rare occasions, the infection invades the surrounding soft tissue and bone; this is known as malignant (necrotizing) otitis externa, and is a medical emergency that occurs primarily in older patients with diabetes mellitus. Otitis externa lasting three months or longer, known as chronic otitis externa, is often the result of allergies, chronic dermatologic conditions, or inadequately treated acute otitis externa.

Otitis media (OM) is one of the most common ear diseases affecting humans. Children are at greater risk and suffer most frequently from OM, which can cause serious deterioration in the quality of life. OM is generally classified into two main types: acute and chronic OM (AOM and COM). AOM is characterized by tympanic membrane swelling or otorrhea and is accompanied by signs or symptoms of ear infection. In COM, there is a tympanic membrane perforation and purulent discharge. Pseudomonas aeruginosa is one of the most common causative agent of otitis media. Pseudomonas aeruginosa is also one of the most common causative agent of otitis interna.

The presence of Pseudomonas aeruginosa can be determined based on the presence of pyocyanin, which is a unique, redox-active chemical marker specific for Pseudomonas aeruginosa. However, many common Pseudomonas aeruginosa infections require diagnosis using samples such as a mixture of cerumen (ear wax), pus, ear exudate, which cannot be used in common diagnostic tests that use the lateral flow platform. The methods disclosed herein allow detection of pyocyanin (or other quorum sensing molecules) in such samples.

Methods of Detecting Specific Bacteria

Accordingly, in some aspects, the present disclosure provides a method for detecting an infection in a subject's ear, comprising, (a) obtaining a sample of cerumen, (b) extracting the cerumen from the applicator, and (c) measuring a presence, absence or amount of a compound in the cerumen, wherein the compound may be redox-active and associated with the infection (e.g. a quorum sensing molecule like pyocyanin). In some embodiments, the measuring comprises contacting the compound with an electrochemical sensor comprising a working electrode and a reference electrode, and electrochemically measuring a current flow. In some embodiments, the current flow is correlated with the presence, absence or amount of the compound. In some embodiments, the cerumen is adsorbed onto an applicator. In some embodiments, the extraction comprises contacting the applicator with a solvent.

In some embodiments, the method further comprises estimating a number of viable cells of a microorganism associated with the infection based on the presence, absence or amount of the compound determined in step (c). In some embodiments, the method detects an electrochemical reaction taking place at the working electrode and thereby quantifies the amount of the compound (e.g. a quorum sensing molecule such as pyocyanin). Accordingly, in these embodiments, the method facilitates an electrochemical detection of the compound (e.g. a quorum sensing molecule such as pyocyanin).

In some embodiments, the cerumen is exuded from the subject's ear. In some embodiments, the cerumen is obtained from the subject's ear canal. In some embodiments, the cerumen is removed from the subject's ear canal using an applicator. Any kind of applicator suitable for removing cerumen may be used. In some embodiments, the applicator is a sterile swab. In some embodiments, the adsorbent portion of the sterile swab is substantially cotton, substantially foam, substantially calcium alginate, substantially nylon, substantially polyester, substantially polyethylene, substantially flocked polyester, or substantially rayon. In some embodiments, the adsorbent portion of the sterile swab is substantially cotton. In some embodiments, the adsorbent portion of the sterile swab is substantially foam. In some embodiments, the applicator is a sterile curette.

In some embodiments, the subject's ear is pre-treated with one or more compositions for softening cerumen before exudation. Several commercial products contain carbamide peroxide (6.5%) in an anhydrous glycerin vehicle as defined in the FDA monograph 21 CFR § 344. Examples of these products are DEBROX Earwax Removal Aid, MURINE Ear Wax Removal Drops, and FLENTS Earwax Removal Aid. Another product is CERUMENEX Eardrops, a prescription product containing triethanolamine polypeptide oleate-condensate (10%). Other compositions for softening cerumen include glycerin (glycerol), olive oil, almond oil, mineral oil, sodium carbonate, sodium bicarbonate, hydrogen peroxide, docusate sodium, and dichlorobenzene.

In some embodiments, the cerumen is extracted from the applicator by contacting the applicator with a solvent. In some embodiments, the solvent is an aqueous solution. In some embodiments, the aqueous solution comprises ingredients selected from salts, buffering agents, and chelating agents. In some embodiments, the solvent may be saline. In some embodiments, the solvent comprises an organic solvent selected from aliphatic hydrocarbons, cyclic hydrocarbons, aromatic hydrocarbons, halogenated hydrocarbons, ketones, amines, esters, alcohols, aldehydes, and ethers. In embodiments, the solvent comprises an organic solvent selected from acetonitrile, alcohol (without limitations, e.g., methanol, ethanol, and isopropanol), acetone, chloroform, dimethyl sulfoxide (DMSO), and dimethylformamide (DMF). In some embodiments, the solvent comprises ethanol or an aqueous solution thereof. In some embodiments, the solvent comprises about 1%, or about 2.5%, or about 5%, or about 7.5%, or about 10%, or about 12.5%, or about 15%, or about 17.5%, or about 20%, or about 25% ethanol.

In embodiments, the solvent comprises an aqueous solution and an alcohol. In embodiments, the solvent comprises a solution made to a physiological pH and isotonic salt concentration. In embodiments, the solvent comprises a Ringer solution. In embodiments, the solvent comprises a balanced salt solution (BSS). In embodiments, the solvent comprises a BSS such as Hanks' Balanced Salt Solution, Earle's Balanced Salt Solution, Dulbeccoo's Balanced Salt Solution, Gey's Balanced Salt Solution, Puk's Saline A, and Krebs-Ringer Bicarbonate Buffer.

In embodiments, the aqueous solution is saline. In embodiments, the saline is buffered. In embodiments, the saline is buffered with a buffer selected from Tris, phosphate, glycine, citrate, HEPES, and glycine. In embodiments, the saline is selected from normal saline (0.90% w/v of NaCl), phosphate buffered saline (PBS), Tris buffered saline (TBS), and Dulbecco's phosphate buffered saline (DPBS).

In embodiments, the solvent comprises a saline and an alcohol. In embodiments, the amount of alcohol present in the solvent is at least about 0.1%, or at least about 0.2%, or at least about 0.5%, or at least about 0.8%, or at least about 1%, or at least about 1.5%, or at least about 2%, or at least about 2.5%, or at least about 3%, or at least about 3.5%, or at least about 4%, or at least about 4.5%, or at least about 6%, or at least about 6.5%, or at least about 7%, or at least about 7.5%, or at least about 8%, or at least about 8.5%, or at least about 9%, or at least about 9.5%, or at least about 10%. In embodiments, the amount of alcohol present in the solvent is less than about 50%, or less than about 40%, or less than about 30%, or less than about 25%, or less than about 20%, or less than about 15%, or less than about 12.5%, or less than about 10%, or less than about 9%, or less than about 8%, or less than about 7.5%, or less than about 7%, or less than about 6.5%, or less than about 6%, or less than about 5.5%, or less than about 5%, or less than about 4.5%, or less than about 4%, or less than about 3.5%, or less than about 3%.

In embodiments, the solvent comprises a saline comprising about 0.1% to about 25%, or about 0.2% to about 20%, or about 0.5% to about 15%, or about 1% to about 10%, or about 2% to about 8%, or about 2.5% to about 7.5%, or about 3% to about 7%, or about 4% to about 6% alcohol.

In embodiments, the solvent comprises a saline comprising about 5% alcohol. In embodiments, the solvent comprises an alcohol selected from methyl alcohol (methanol), ethyl alcohol (ethanol), isopropyl alcohol (isopropanol), and butyl alcohol (butanol). In embodiments, the solvent comprises methanol. In embodiments, the solvent comprises ethanol. In embodiments, the solvent comprises a saline and at least about 0.1%, or at least about 0.2%, or at least about 0.5%, or at least about 0.8%, or at least about 1%, or at least about 1.5%, or at least about 2%, or at least about 2.5%, or at least about 3%, or at least about 3.5%, or at least about 4%, or at least about 4.5%, or at least about 6%, or at least about 6.5%, or at least about 7%, or at least about 7.5%, or at least about 8%, or at least about 8.5%, or at least about 9%, or at least about 9.5%, or at least about 10% ethanol. In embodiments, the solvent comprises a saline and less than about 50%, or less than about 40%, or less than about 30%, or less than about 25%, or less than about 20%, or less than about 15%, or less than about 12.5%, or less than about 10%, or less than about 9%, or less than about 8%, or less than about 7.5%, or less than about 7%, or less than about 6.5%, or less than about 6%, or less than about 5.5%, or less than about 5%, or less than about 4.5%, or less than about 4%, or less than about 3.5%, or less than about 3% ethanol.

In embodiments, the solvent comprises a phosphate buffered saline (PBS) and an alcohol. In embodiments, the alcohol is ethanol. In embodiments, the solvent comprises PBS comprising ethanol in an amount of about 0.1% to about 25%, or about 0.2% to about 20%, or about 0.5% to about 15%, or about 1% to about 10%, or about 2% to about 8%, or about 2.5% to about 7.5%, or about 3% to about 7%, or about 4% to about 6%. In embodiments, the solvent comprises PBS comprising about 5% ethanol. In embodiments, the solvent further comprises about 1 mM MgCl₂.

In embodiments, the solvent comprises a divalent cation selected from Ba⁺², Be⁺², Cd⁺², Ca⁺², Co⁺², Cu⁺², Ga⁺², Fe, Mg⁺², and Z⁺². In embodiments, the divalent cation is present at a concentration of about 0.1 mM to about 5 mM, or about 0.25 mM to about 3 mM, or about 0.5 mM to about 2 mM, or about 0.75 mM to about 1.25 mM. In embodiments, the divalent cation is present at a concentration of about 1 mM.

In embodiments, the solvent comprises Mg⁺². In embodiments, the solvent comprises about 0.1 mM to about 5 mM, or about 0.25 mM to about 3 mM, or about 0.5 mM to about 2 mM, or about 0.75 mM to about 1.25 mM Mg⁺². In embodiments, the solvent comprises about 1 mM Mg⁺². In embodiments, the solvent comprises about 0.1 mM to about 5 mM, or about 0.25 mM to about 3 mM, or about 0.5 mM to about 2 mM, or about 0.75 mM to about 1.25 mM MgCl₂ or MgSO₄. In embodiments, the solvent comprises about 1 mM MgCl₂ or MgSO₄.

In embodiments, the solvent comprises a phosphate buffered saline (PBS) comprising about 0.1% to about 25%, or about 0.2% to about 20%, or about 0.5% to about 15%, or about 1% to about 10%, or about 2% to about 8%, or about 2.5% to about 7.5%, or about 3% to about 7%, or about 4% to about 6% ethanol and about 0.1 mM to about 5 mM, or about 0.25 mM to about 3 mM, or about 0.5 mM to about 2 mM, or about 0.75 mM to about 1.25 mM Mg⁺². In embodiments, the solvent comprises a phosphate buffered saline (PBS) comprising about 5% ethanol and about 1 mM Mg⁺². In embodiments, the solvent comprises a phosphate buffered saline (PBS) comprising about 5% ethanol and about 1 mM MgCl₂ or MgSO₄. In embodiments, the solvent comprises a phosphate buffered saline (PBS) comprising about 5% ethanol and about 1 mM MgCl₂.

In some embodiments, the solvent further comprises a cerumenolytic. In some embodiments, the cerumenolytic is selected from dioctyl sodium sulfosuccinate (DOSS), dioctyl calcium sulfosuccinate, urea, sodium bicarbonate, acetic acid, almond oil, peanut oil, rectified camphor oil, olive oil, mineral oil, liquid petrolatum, docusate sodium, triethanolamine polypeptide oleate-condensate, choline salicylate and glycerin.

In some embodiments, the extracting removes a substantial amount of the cerumen from the applicator. In some embodiments, the extracting removes a substantial amount of the compound (e.g. a quorum sensing molecule such as pyocyanin) from the cerumen. In some embodiments, the applicator is contacted with the solvent in a collection tube. In some embodiments, the extracting occurs in the collection tube. In some embodiments, the extracting occurs on the surface of the electrochemical sensor. In some embodiments, the extracting occurs on a hydrophilic membrane attached to or contacted with the electrochemical sensor. In some embodiments, the extracting occurs on a hydrophilic membrane that is adjoined to the working electrode and/or the reference electrode. In some embodiments, the hydrophilic membrane wicks the cerumen from the applicator. In some embodiments, the hydrophilic membrane sorbs (e.g. absorbs or adsorbs) the solvent.

In some embodiments, the concentration of the compound (e.g. a quorum sensing molecule such as pyocyanin) is measured by introducing a fluid sample extracted from the applicator onto the electrochemical sensor including a working electrode and a reference electrode; performing an electrochemical measurement to detect the compound (e.g. a quorum sensing molecule such as pyocyanin) in the fluid sample extracted from the applicator determining a concentration of the compound (e.g. a quorum sensing molecule such as pyocyanin) in the fluid sample extracted from the applicator by using a previously determined correlation between known concentrations of the compound (e.g. a quorum sensing molecule such as pyocyanin) and a current flow through the working electrode. In some embodiments, the defined potential of the working electrode may be varied, and the response from the electrochemical reaction is seen from the current of the working electrode.

In some embodiments, the compound detected is a quorum sensing molecule. In some embodiments, the quorum sensing molecule is a phenazine compound. In some embodiments, the phenazine compound is pyocyanin. In some embodiments, the presence of pyocyanin is indicative of the presence or extent of Pseudomonas aeruginosa infection.

In some embodiments, the infection is a Pseudomonas aeruginosa infection. In some embodiments, the infection is one or more of otitis externa, otitis media, and otitis interna.

In some embodiments, a given quorum sensing molecule (e.g., without limitation, pyocyanin) electrochemically reacts differently on different electrode surfaces. Thus, different electrode materials and geometries used for chemical detection will give different results. Accordingly, in some embodiments, the sensor array increases the sensitivity and specificity of the measurement, and reduces the noise from other substances present in a biological sample.

In some embodiments, the electrochemical sensor used in the method disclosed herein comprises a second working electrode. In some embodiments, the second working electrode differs from the first electrode with respect to one or more of surface area, size, material, and coating. In some embodiments, the electrochemical sensor used in the method disclosed herein may include an oxidizing working electrode and a reducing working electrode. In some embodiments, the concentration of a predetermined quorum sensing molecule (e.g., without limitation, pyocyanin) is measured as current flow through the oxidizing electrode and the reducing electrode. In some embodiments, the working electrode is one of an oxidizing electrode and a reducing electrode, and the second working electrode is the other of the oxidizing electrode and the reducing electrode. A potential suitable for oxidizing the predetermined quorum sensing molecule (e.g., without limitation, pyocyanin) is applied at the oxidizing electrode and a potential suitable for reducing the predetermined quorum sensing molecule (e.g., without limitation, pyocyanin) is applied at the reducing electrode. In some embodiments, the electrochemical sensor comprises a second working electrode and the working electrode is one of an oxidizing electrode and a reducing electrode, and the second working electrode is the other of the oxidizing electrode and the reducing electrode.

In some embodiments, if the current flow through the working electrode is less than about 10 nA, the microorganism associated with the infection is considered nonviable or absent. In some embodiments, the method is capable of detecting a current flow through the working electrode of at least about 0.5 nA, or at least about 1 nA, or at least about 2.5 nA, or at least about 5 nA, or at least about 0.01 μA, or at least about 0.02 μA, or at least about 0.05 μA, or at least about 0.1 μA, or at least about 0.2 μA, or at least about 0.5 μA, or at least about 1 μA. In some embodiments, the current flow through the working electrode is more than about 10 nA, or more than about 25 nA, or more than about 50 nA, or more than about 100 nA, or more than about 250 nA, or more than about 0.5 μA, or more than about 1 μA, or more than about 2 μA, more than about 4 μA, or more than about 6 μA, more than about 8 μA, or more than about 10 μA, more than about 15 μA, or more than about 20 μA, more than about 50 μA, or more than about 75 μA, more than about 100 μA, the microorganism associated with the infection is considered present. In some embodiments, the current flow through the working electrode is more than about 10 nA, or more than about 25 nA, or more than about 50 nA, or more than about 100 nA, or more than about 250 nA, or more than about 0.5 μA, or more than about 1 μA, or more than about 2 μA, more than about 4 μA, or more than about 6 μA, more than about 8 μA, or more than about 10 μA, more than about 15 μA, or more than about 20 μA, more than about 50 μA, or more than about 75 μA, more than about 100 μA, a chronic infection by the microorganism associated with the infection is indicated. In some embodiments, the current flow through the working electrode is more than about 10 nA, or more than about 25 nA, or more than about 50 nA, or more than about 100 nA, or more than about 250 nA, or more than about 0.5 μA, or more than about 1 μA, or more than about 2 μA, more than about 4 μA, or more than about 6 μA, more than about 8 μA, or more than about 10 μA, more than about 15 μA, or more than about 20 μA, more than about 50 μA, or more than about 75 μA, more than about 100 μA, an acute infection by the microorganism associated with the infection is indicated. In some embodiments, the current flow through the working electrode is more than about 10 nA, or more than about 25 nA, or more than about 50 nA, or more than about 100 nA, or more than about 250 nA, or more than about 0.5 μA, or more than about 1 μA, or more than about 2 μA, more than about 4 μA, or more than about 6 μA, more than about 8 μA, or more than about 10 μA, more than about 15 μA, or more than about 20 μA, more than about 50 μA, or more than about 75 μA, more than about 100 μA, a biofilm infection by the microorganism associated with the infection is indicated.

In some embodiments, the method detects peak height from the baseline of the curve. In some embodiments, the baseline of the curve is at 0. In some embodiments, the baseline of the curve is not at 0. In some embodiments, peak height is measured. In some embodiments, the peak height is controlled by variables selected from concentration of the analyte, buffer, contaminants, if any, the electrode material, electrode size, frequency of electrochemical scan, amplitude of the squarewave, and step size. In some embodiments, the location of the peak determines identity of the redox molecule (without limitation, e.g. pyocyanin). Accordingly, in some embodiments, the limit of detection of an analyte (without limitation, e.g. pyocyanin) is limited by the signal to noise ratio. For example, the concentration of pyocyanin that can be detected depends on the electrode material. The electrode size affects both the pyocyanin signal and the measured noise/background.

In some embodiments, the method detects less than about 0.1 nM, or less than about 0.25 nM, or less than about 0.5 nM, or less than about 1 nM, or less than about 2.5 nM, or less than about 5 nM, or less than about 10 nM, or less than about 25 nM, or less than about 50 nM, or less than about 100 nM, or less than about 250 nM, or less than about 500 nM, or less than about 1 μM, or less than about 5 μM, or less than about 10 μM, or less than about 20 μM, or less than about 30 μM, or less than about 40 μM, or less than about 50 μM, or less than about 100 μM, or less than about 200 μM pyocyanin. In some embodiments, the method includes providing an indication of a presence of Pseudomonas aeruginosa when the concentration of the detected pyocyanin is above at least 1 nM, or at least 2.5 nM, or at least 5 nM, or at least 10 nM, or at least 25 nM, or at least 50 nM, or at least 100 nM, or at least 250 nM, or at least 500 nM, or at least 1 μM, or at least 2 μM, or at least 5 μM, or at least 10 μM, or greater.

In some embodiments, the concentration of the detected pyocyanin is above at least 1 nM, or at least 2.5 nM, or at least 5 nM, or at least 10 nM, or at least 25 nM, or at least 50 nM, or at least 100 nM, or at least 250 nM, or at least 500 nM, or at least 1 μM, or at least 2 μM, or at least 5 μM, or at least 10 μM, the microorganism associated with the infection is considered present. In some embodiments, the concentration of the detected pyocyanin is above at least 1 nM, or at least 2.5 nM, or at least 5 nM, or at least 10 nM, or at least 25 nM, or at least 50 nM, or at least 100 nM, or at least 250 nM, or at least 500 nM, or at least 1 μM, or at least 2 μM, or at least 5 μM, or at least 10 μM, a chronic infection by the microorganism associated with the infection is indicated. In some embodiments, the concentration of the detected pyocyanin is above at least 1 nM, or at least 2.5 nM, or at least 5 nM, or at least 10 nM, or at least 25 nM, or at least 50 nM, or at least 100 nM, or at least 250 nM, or at least 500 nM, or at least 1 μM, or at least 2 μM, or at least 5 μM, or at least 10 μM, an acute infection by the microorganism associated with the infection is indicated.

In some embodiments, the electrochemical measurement is selected from square wave voltammetry, linear sweep voltammetry, staircase voltammetry, cyclic voltammetry, normal pulse voltammetry, differential pulse voltammetry, and chronoamperometry. In some embodiments, the electrochemical measurement is square wave voltammetry and the current flow is measured in response to one or more square wave potentials.

In some embodiments, the presence, absence or amount of the compound is measured as current flow through the working electrode. In some embodiments, the presence, absence or amount of compound is measured as current flow through the oxidizing electrode and the reducing electrode.

In some embodiments, the method informs the administration of one or more antibiotics upon a positive test for infection. In some embodiments, the current flow through the working electrode of more than about 10 nA, or more than about 25 nA, or more than about 50 nA, or more than about 100 nA, or more than about 250 nA, or more than about 0.5 μA, or more than about 1 μA, or more than about 2 μA, more than about 4 μA, or more than about 6 μA, more than about 8 μA, or more than about 10 μA, more than about 15 μA, or more than about 20 μA, more than about 50 μA, or more than about 75 μA, more than about 100 μA constitutes a positive test for infection, which informs the administration of one or more antibiotics. In some embodiments, the method informs the withholding of one or more antibiotics upon a negative test for infection. In some embodiments, the current flow through the working electrode of more than about 10 nA, or more than about 25 nA, or more than about 50 nA, or more than about 100 nA, or more than about 250 nA, or more than about 0.5 μA, or more than about 1 μA, or more than about 2 μA, more than about 4 μA, or more than about 6 μA, more than about 8 μA, or more than about 10 μA, more than about 15 μA, or more than about 20 μA, more than about 50 μA, or more than about 75 μA, more than about 100 μA informs the withholding of one or more antibiotics and/or changing dose, regimen and/or combination of one or more antibiotics. In some embodiments, the current flow through the working electrode of less than about 0.1 μA, or less than about 0.2 μA, less than about 0.4 μA, or less than about 0.6 μA, less than about 0.8 μA, or less than about 1.0 μA, less than about 1.5 μA, or less than about 2.0 μA, less than about 5 μA, or less than about 7.5 μA, less than about 10 μA constitutes a negative test for infection, which informs the withholding of one or more antibiotics and/or changing dose, regimen and/or combination of one or more antibiotics. In some embodiments, the method informs the selection of an appropriate antibiotic for the infectious agent upon a positive test for infection. In some embodiments, the current flow through the working electrode of more than about 10 nA, or more than about 25 nA, or more than about 50 nA, or more than about 100 nA, or more than about 250 nA, or more than about 0.5 μA, or more than about 1 μA, or more than about 2 μA, more than about 4 μA, or more than about 6 μA, more than about 8 μA, or more than about 10 μA, more than about 15 μA, or more than about 20 μA, more than about 50 μA, or more than about 75 μA, more than about 100 μA informs the selection of an appropriate antibiotic for the infectious agent upon a positive test for infection.

In some embodiments, the fluid sample extracted from an applicator may be introduced continuously into the electrochemical sensor. In some embodiments, the fluid samples extracted from applicators are repeatedly introduced into the electrochemical sensor. In some embodiments, the fluid samples extracted from applicators are introduced only once into the electrochemical sensor. For example, the steps of introducing a fluid sample extracted from an applicator into the device, performing an electrochemical measurement to detect a redox-active compound associated with the infection (e.g., without limitation, pyocyanin) in the fluid sample extracted from an applicator, and determining a concentration of the redox-active compound associated with the infection in the fluid sample can be performed repeatedly at time intervals. In some embodiments, the steps can be repeated at least every 6 hours, every 12 hours, every 18 hours, every 24 hours, or every 48 hours.

In other embodiments, the fluid sample extracted from an applicator can be introduced into a well, chamber, or another form of receptacle in which the reaction can take place. The volume of the channel, well, chamber or other receptacle can be less than about 50 nanoliters (nL), less than about 10 nL, less than about 1 nL, less than about 100 picoliters (pL), less than about 50 pL, less than about 10 pL, less than about 5 pL, or less than about 1 pL.

In some embodiments, a sample volume that is introduced into the electrochemical sensor can be less than about 100 μL, less than about 50 μL, less than about 20 μL, less than about 10 μL, less than about 5 μL, less than about 2 μL, or less than about 1 μL.

In some embodiments, a capillary or wicking material can be disposed at or near an inlet of the electrochemical sensor to draw the fluid sample into the device. Is some embodiments, a matrix material can be disposed at or near an inlet of an electrochemical sensor, for example, to isolate the electrodes from the bacteria while permitting passage of pyocyanin to access the electrodes.

In some embodiments, the detection of quorum sensing molecules provides information about the presence of infection by a specific bacterial species (e.g. Pseudomonas aeruginosa) and severity of the infection based on the amount of a quorum sensing molecule (e.g., without limitation pyocyanin) in a sample (e.g. cerumen). For example, in some embodiments, the detection of less than about 0.1 nM, or less than about 0.25 nM, or less than about 0.5 nM, or less than about 1 nM, or less than about 2.5 nM, or less than about 5 nM, or less than about 10 nM, or less than about 25 nM, or less than about 50 nM, or less than about 100 nM, or less than about 250 nM, or less than about 500 nM, or less than about 1 μM, or less than about 5 μM, or less than about 10 μM, or less than about 20 μM, or less than about 30 μM, or less than about 40 μM, or less than about 50 μM, or less than about 100 μM pyocyanin provides information about the presence of infection Pseudomonas aeruginosa. In some embodiments, the detection of less than about 1 μM, or less than about 5 μM, or less than about 10 μM, or less than about 20 μM, or less than about 30 μM, or less than about 40 μM, or less than about 50 μM, or less than about 100 μM pyocyanin informs a chronic Pseudomonas aeruginosa infection. In some embodiments, the detection of less than about 1 μM, or less than about 5 μM, or less than about 10 μM, or less than about 20 μM, or less than about 30 μM, or less than about 40 μM, or less than about 50 μM, or less than about 100 μM pyocyanin informs an acute Pseudomonas aeruginosa infection.

In some embodiments, a number of viable cells of specific bacteria (e.g. Pseudomonas aeruginosa) in the biofilm can be estimated based on the determined concentration of a quorum sending molecule (e.g., without limitation, pyocyanin) in a sample (e.g. cerumen). In some embodiments, (e.g. if the current flow through the working electrode is less than about 1 μA), the detection of lower than a threshold amount of a quorum sending molecule (e.g., without limitation, pyocyanin) in a sample (e.g. cerumen) may indicate that the sample contains no specific bacteria or contains nonviable specific bacteria (e.g. Pseudomonas aeruginosa).

In some embodiments, a given predetermined quorum sensing molecule (e.g., without limitation, pyocyanin) electrochemically reacts on the electrode surfaces providing specific current output dependent on variables such as surface area, size, material, and coating of the electrode. In some embodiments, the characteristics of the current output at a well-defined potential may be experimentally or theoretically determined and used for specific identification and optionally quantitation of the given predetermined quorum sensing molecule (e.g., without limitation, pyocyanin). Accordingly, in some embodiments, the method disclosed herein is used for measuring an electrochemical reaction taking place due to the predetermined quorum sensing molecule (e.g., without limitation, pyocyanin) in a sample (e.g. cerumen) at the working electrode at a well-defined potential. The electrochemical sensor thereby facilitates an electrochemical detection of a predetermined quorum sensing molecule (e.g., without limitation, pyocyanin). In some embodiments, the concentration of the predetermined quorum sensing molecule (e.g., without limitation, pyocyanin) is measured by introducing a fluid sample extracted from an applicator into an electrochemical sensor including a working electrode and a reference electrode; performing an electrochemical measurement to detect the predetermined quorum sensing molecule (e.g., without limitation, pyocyanin) in the fluid sample extracted from an applicator determining a concentration of the predetermined quorum sensing molecule (e.g., without limitation, pyocyanin) in the fluid sample extracted from an applicator by using a previously determined correlation between known concentrations of the predetermined quorum sensing molecule (e.g., without limitation, pyocyanin) and a current flow through the working electrode. In some embodiments, the defined potential of the working electrode may be varied, and the response from the electrochemical reaction is seen from the current of the working electrode. In some embodiments, a threshold level of a predetermined quorum sensing molecule (e.g., without limitation, pyocyanin) can be a concentration less than about 0.1 nM, or less than about 0.25 nM, or less than about 0.5 nM, or less than about 1 nM, or less than about 2.5 nM, or less than about 5 nM, or less than about 10 nM, or less than about 25 nM, or less than about 50 nM, or less than about 100 nM, or less than about 250 nM, or less than about 500 nM, or less than about 1 μM, or less than about 5 μM, or less than about 10 μM, or less than about 20 μM, or less than about 30 μM, or less than about 40 μM, or less than about 50 μM, or less than about 100 μM, or less than about 200 μM, or greater. In some embodiments, the method includes providing an indication of a presence of a pathogen (e.g. Pseudomonas aeruginosa) when the concentration of the predetermined quorum sensing molecule (e.g., without limitation, pyocyanin) is above at least 1 μM, at least 2 μM, at least 5 μM, or at least 10 μM, or greater. In some embodiments, the method includes estimating a number of cells of the pathogen (e.g. Pseudomonas aeruginosa) based on the concentration of the predetermined quorum sensing molecule (e.g., without limitation, pyocyanin).

In some embodiments, the method monitors the effectiveness of an antibiotic treatment of an infection (e.g. Pseudomonas aeruginosa infection) in a patient. In some embodiments, the method informs the effectiveness of an antibiotic treatment of an infection (e.g. Pseudomonas aeruginosa infection) in a patient upon a negative test for infection. In some embodiments, the current flow through the working electrode of less than about 0.1 μA, or less than about 0.2 μA, less than about 0.4 μA, or less than about 0.6 μA, less than about 0.8 μA, or less than about 1.0 μA, less than about 1.5 μA, or less than about 2.0 μA, less than about 5 μA, or less than about 7.5 μA, less than about 10 μA informs the effectiveness of an antibiotic treatment of an infection (e.g. Pseudomonas aeruginosa infection) in a patient. In some embodiments, a decrease in the current flow through the working electrode from the method performed after the treatment compared to the method performed before treatment or using a pretreatment sample (e.g. cerumen) informs the effectiveness of an antibiotic treatment of an infection (e.g. Pseudomonas aeruginosa infection) in a patient.

In some embodiments, the method informs the lack of effectiveness of an antibiotic treatment of an infection (e.g. Pseudomonas aeruginosa infection) in a patient upon a positive test for infection. In some embodiments, the current flow through the working electrode of more than about 10 nA, or more than about 25 nA, or more than about 50 nA, or more than about 100 nA, or more than about 250 nA, or more than about 0.5 μA, or more than about 1 μA, or more than about 2 μA, more than about 4 μA, or more than about 6 μA, more than about 8 μA, or more than about 10 μA, more than about 15 μA, or more than about 20 μA, more than about 50 μA, or more than about 75 μA, more than about 100 μA informs the lack of effectiveness of an antibiotic treatment of an infection (e.g. Pseudomonas aeruginosa infection) in a patient. In some embodiments, an increase in the current flow through the working electrode from the method performed after the treatment compared to the method performed before treatment or using a pretreatment sample (e.g. cerumen) informs the lack of effectiveness of an antibiotic treatment of an infection (e.g. Pseudomonas aeruginosa infection) in a patient. In some embodiments, a lack of substantial change in the current flow through the working electrode from the method performed after the treatment compared to the method performed before treatment or using a pretreatment sample (e.g. cerumen) informs the lack of effectiveness of an antibiotic treatment of an infection (e.g. Pseudomonas aeruginosa infection) in a patient.

Subjects

In some embodiments, the subject is a non-human animal. In some embodiments, the non-human animal is a captive animal. In some embodiments, the non-human animal is a pet animal, a farm animal, or a zoo animal. In some embodiments, the subject is a mammal, e.g., dog, cat, horse, cow, pig, rabbit, sheep, mouse, rat, guinea pig or non-human primate, such as a monkey, chimpanzee, or baboon. In some embodiments, the subject is a canine or a feline. In some embodiments, the subject is a dog (i.e. a canine), or other members of the family Canidae.

Kits

In some aspects, the present disclosure provides a kit for detecting an infection in a subject's ear. In some embodiments, the kit comprises (a) an applicator suitable for adsorbing a sample of cerumen and (b) a solvent suitable for extracting the cerumen and/or a compound within the cerumen from the applicator. In some embodiments, the kit comprises (a) an applicator suitable for adsorbing a sample of cerumen; (b) a solvent suitable for extracting the cerumen and/or a compound within the cerumen from the applicator; and (c) an electrochemical sensor, the electrochemical sensor comprising a working electrode and a reference electrode and being suitable for electrochemically measuring a current flow through the sensor, which is correlated with the presence, absence or amount of the compound.

In some embodiments, the electrochemical sensor comprises a working electrode and a reference electrode, and a test strip, comprising a means for receiving the sample. In some embodiments, the electrochemical sensor is connected to a reader that performs the electrochemical measurements. In some embodiments, the reader comprises a potentiostat that applies voltage to the sensor and measures current output. In some embodiments, the current output is used to determine the amount of the compound. In some embodiments, the kit comprises (a) an applicator suitable for adsorbing a sample of cerumen; (b) a solvent suitable for extracting the cerumen and/or a compound within the cerumen from the applicator; and (c) the test strip. In some embodiments, the kit comprises (a) an applicator suitable for adsorbing a sample of cerumen; (b) a solvent suitable for extracting the cerumen and/or a compound within the cerumen from the applicator; (c) the test strip; and (d) the reader. In some embodiments, the kit comprises a plurality of (a) an applicators suitable for adsorbing a sample of cerumen; (b) a solvents suitable for extracting the cerumen and/or a compound within the cerumen from the applicator; and (c) the test strips; and a single (d) reader.

In some embodiments, the kit further comprises one or more of positive control samples, negative control samples, a key for estimating a number of viable cells of a microorganism, and instructions to use. In some embodiments, the kit further comprises a collection tube.

In some embodiments, the applicator is a sterile swab or a sterile curette. In some embodiments, the adsorbent portion of the sterile swab is made of substantially cotton, substantially foam, substantially calcium alginate, substantially nylon, substantially polyester, substantially polyethylene, substantially flocked polyester, or substantially rayon.

In some embodiments, the solvent is an aqueous solution. In some embodiments, the aqueous solution comprises ingredients selected from salts, buffering agents, and chelating agents. In some embodiments, the solvent is saline.

In some embodiments, the solvent is a non-aqueous solution. In some embodiments, the solvent comprises an organic solvent selected from aliphatic hydrocarbons, cyclic hydrocarbons, aromatic hydrocarbons, halogenated hydrocarbons, ketones, amines, esters, alcohols, aldehydes, and ethers. In some embodiments, the solvent comprises ethanol or an aqueous solution thereof. In some embodiments, the solvent comprises about 1%, or about 2.5%, or about 5%, or about 7.5%, or about 10%, or about 12.5%, or about 15%, or about 17.5%, or about 20%, or about 25% ethanol.

In some embodiments, the solvent further comprises a cerumenolytic. In some embodiments, the cerumenolytic is selected from dioctyl sodium sulfosuccinate (DOSS), dioctyl calcium sulfosuccinate, urea, sodium bicarbonate, acetic acid, almond oil, peanut oil, rectified camphor oil, olive oil, mineral oil, liquid petrolatum, docusate sodium, triethanolamine polypeptide oleate-condensate, choline salicylate and glycerin

In some embodiments, the positive control comprises Pseudomonas aeruginosa cells or a metabolite thereof. In some embodiments, the positive control comprises a quorum sensing molecule. In some embodiments, the quorum sensing molecule is a phenazine compound. In some embodiments, the phenazine compound is pyocyanin.

In some embodiments, the electrochemical sensor generates a waveform suitable for performing electrochemical measurement selected from the group consisting of square wave voltammetry, linear sweep voltammetry, staircase voltammetry, cyclic voltammetry, normal pulse voltammetry, differential pulse voltammetry, and chronoamperometry. In some embodiments, the electrochemical sensor generates a waveform suitable for performing square wave voltammetry, wherein the current flow is measured in response to one or more square wave potentials.

In some embodiments, the electrochemical sensor comprises a second working electrode and the working electrode is one of an oxidizing electrode and a reducing electrode, and the second working electrode is the other of the oxidizing electrode and the reducing electrode. In some embodiments, the working electrode is comprised of gold (Au), silver (Ag), platinum (Pt), indium tin oxide (ITO), carbon, carbon nanotubes, carbon nanofibers, graphene, carbon-platinum composites, carbon nanotubes with gold nanoparticles, and any combination thereof. In some embodiments, the reference electrode is comprised of silver (Ag), silver chloride (AgCl), gold (Au), palladium (Pd), and platinum (Pt), and any combination thereof.

### EXAMPLES

The examples herein are provided to illustrate advantages and benefits of the present technology and to further assist a person of ordinary skill in the art with using the methods and preparing the kits of the present technology. The examples herein are also presented in order to more fully illustrate the preferred aspects of the present technology. The examples should in no way be construed as limiting the scope of the present disclosure, as defined by the appended claims. The examples can include or incorporate any of the variations, aspects or embodiments of the present technology described above. The variations, aspects or embodiments described above may also further each include or incorporate the variations of any or all other variations, aspects or embodiments of the present technology.

### Example 1. Sample Collection and the System for the Diagnosis

Veterinarians use a cotton swab to obtain samples from the ear of a dog for diagnostic purposes. Sample was collected using a cotton swab from a dog's ear. As shown in FIG. 1, the swab collects cerumen and exudate and wax. Similar technique may be used for sample collection from other animals or humans. Downstream processing requires minimal to no sample preparation once the swab sample is collected from the dog's ear.

These results indicate that sample collection with a swab represents a simple method for sample collection.

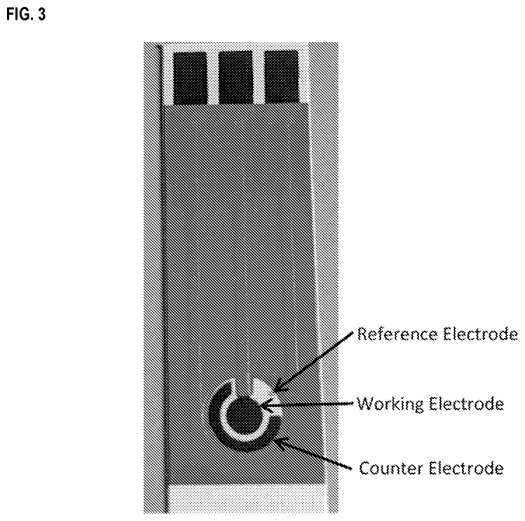

Electrochemical diagnostic tests can include a number of different voltammetry or impedance based scanning techniques. The sensor described herein uses voltammetry where peak currents are measured at particular applied voltages (FIG. 3). The sensor disclosed herein comprises three electrodes: a working electrode, a counter electrode, and a reference electrode. Electrochemical tests using the sensor disclosed herein require that all electrodes on the sensor (working, counter, and reference electrodes) be fully contacted by the same electrically conductive liquid to promote electron transfer. Thus, the electrochemical sensors used for this study require at least 50-100 μL of liquid sample to fully wet all of the electrodes. To measure peak currents with a standard, 100 μL of saline solution spiked with redox molecule was pipetted onto the sensor to fully wet all the electrodes simultaneously. An electrochemical scan was taken. As shown in FIG. 3, the electrochemical scan with the saline solution spiked with redox molecule showed a peak current located at −0.29 V.

These results demonstrate that the sensor disclosed herein is suitable for the detection of redox molecules via an electrochemical scan.

To improve sample collection and performing reliable tests, the system was tested using dog ear wax and exudate on cotton swabs.

### Example 2. Detection of Redox Molecules Following Directly Rubbing the Exudate from Cotton Swab onto the Electrochemical Sensor

A simple way to transfer the material from the swab onto the sensor is to directly rub the swab onto the sensor. If the sample contains enough liquid, it may be able to wet the entire sensor area and contact all electrodes simultaneously. Therefore, whether this method is suitable for the detection of redox molecules using the sensor disclosed herein was explored.

It was observed that hardly any exudate fluid came out of the swab, just some wax ended up being transferred. Enough liquid material was not transferred onto the sensor to easily and totally cover all of the electrodes.

These results indicate that it was still possible that there was enough exudate liquid in the swab but the transfer onto the sensor was not efficient.

### Example 3. Recovery of the Exudate from a Cotton Swab by Centrifugation

Although directly rubbing the swab onto the sensor did not sufficiently transfer the exudate onto the sensor to easily and totally cover all of the electrodes, it was still possible that there was enough exudate liquid in the swab but the transfer onto the sensor was not efficient, and that the exudate liquid needs to be removed through force, e.g. by centrifugation. Therefore, the cotton swab was centrifuged to see if enough exudate liquid would be released and can be pipetted onto the sensor. The swabs were inserted into 1.5 mL microcentrifuge tubes, the stick of the swab was cut to fit the swab in the microcentrifuge tubes with the cap closed and centrifuged at 10,000 rpm for 5 minutes.

No liquid collected in the tube after centrifugation. The centrifugation did not help to transfer any liquid from the sample to the sensor.

These results indicate that if there was not enough liquid to be used directly in the swab sample, the protocol may require adding in some liquid to help dissolve the wax and exudate so it can be transferred to the electrode surface. It may still be possible to extract the liquid using specialized tubes that hold swab material while allowing the centrifugal extraction of the fluid.

### Example 4. Extraction of the Exudate from a Cotton Swab Using a Liquid

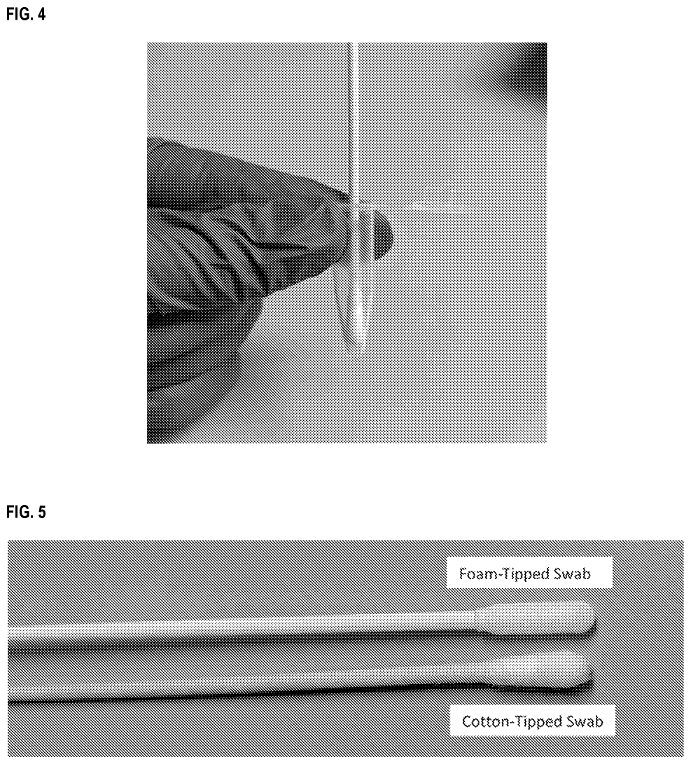

To test whether adding in some liquid to help dissolve the wax and exudate would be helpful to obtain sufficient liquid for covering all of the electrode surfaces, the swab was placed in 50 μL of saline solution in a microcentrifuge tube for 1 minute. As shown in FIG. 4, the swab immediately absorbed all of the saline. The swab was rubbed onto the electrode. The swab was then cut to fit with the cap closed and spun in a centrifuge.

The cotton swab absorbed all of the 50 μL of solution. The added saline could not be extracted by direct transfer of rubbing the swab onto the electrode. Collecting the fluid by a centrifugation process disclosed in Example 3 also resulted in no recovery of the solution. Trying to directly pipette the solution out of the cotton swab also proved to be unsuccessful.

These results indicate that cotton swab absorbs 50 μL of solution.

### Example 5. Extraction of the Exudate from a Cotton Swab Using Larger Volume of a Liquid

Since the swab absorbed all of the 50 μL of solution, the protocol of Example 4 was attempted with a larger volume of liquid, 100 μL of saline solution.

Again the swab absorbed and held onto all of the solution.

These results indicate that extracting sample with a liquid more than about 100 μL would dilute the ear swab sample too much for accurate diagnosis. Ultimately, the traditional cotton swabs are too absorbent and this particular transfer technique requires a different swab material.

### Example 6. Testing the Transfer of Ear Wax and Exudate Material Using a Foam Swab

Since the cotton swab was found to be too absorbent, a different swab with a foam tip was used in a similar set of material transferring experiments outlined below. As shown in FIG. 5, the new swab (upper swab) has a foamy/spongy texture, made of polyurethane foam, compared to the cotton swab that is a wrapped bundle of cotton (lower swab). Transfer of the ear wax/exudate from the foam swab was attempted by directly rubbing the swab onto the sensor.

Although the material transferred liquid more easily from the foam compared to the cotton swab, there was still not enough liquid material to cover the entire sensor surface.

These results indicate that transfer of the ear wax/exudate from the foam swab is possible but would be reliable when the sample contains lots of exudate.

### Example 7. Recovery of the Exudate from a Foam Swab by Centrifugation

It was still possible that there was enough exudate liquid in the foam swab but the transfer onto the sensor was not efficient, and that the exudate liquid needs to be removed through force, e.g. by centrifugation. Therefore, the foam swabs were inserted into 1.5 mL microcentrifuge tubes, the stick of the swab was cut to fit the swab in the microcentrifuge tubes with the cap closed and centrifuged at 10,000 rpm for 5 minutes.

Centrifuging the foam-tipped swab did not result in any liquid collection in the microcentrifuge tube.

These results indicate that centrifugation did not help to transfer any liquid from the sample to the sensor.

### Example 8. Extraction of the Exudate from a Foam Swab Using a Liquid

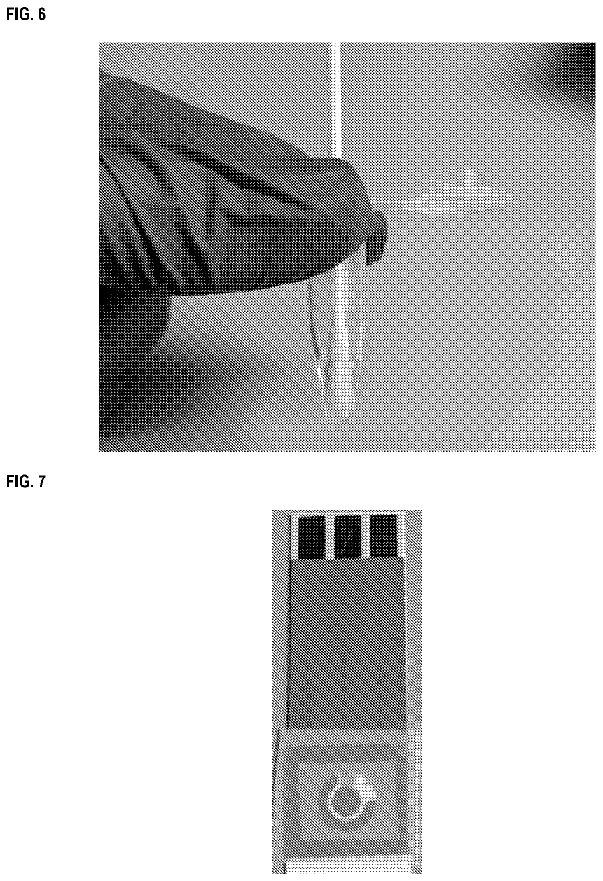

Next, whether adding in some liquid to help dissolve the wax and exudate would be helpful to obtain sufficient liquid for covering all of the electrode surfaces was tested. The foam-tipped swab was placed in 100 μL of saline solution to see if wax material transfers from the swab to the solution and does not all absorb into the swab. As shown in FIG. 6, unlike the cotton swab, the foam swab did not absorb the saline.

When the foam swab was placed in 100 μL of saline, the wax and exudate dissolved into the solution and not all of the solution was absorbed back into the swab. There was enough liquid left in the vial to pipette onto the sensor, but that involved an extra piece of equipment to perform the material transfer. When removing the swab from the saline solution, it still appeared wet and could be rubbed onto the sensor.

These results indicate that this setup resulted in the most liquid transfer, but is still not enough liquid to cover all of the electrodes evenly and fully. Overall, the foam-tipped swab worked better for releasing the wax and exudate material from the swab into a saline solution.

### Example 9. Covering the Sensor with a Hydrophilic Membrane

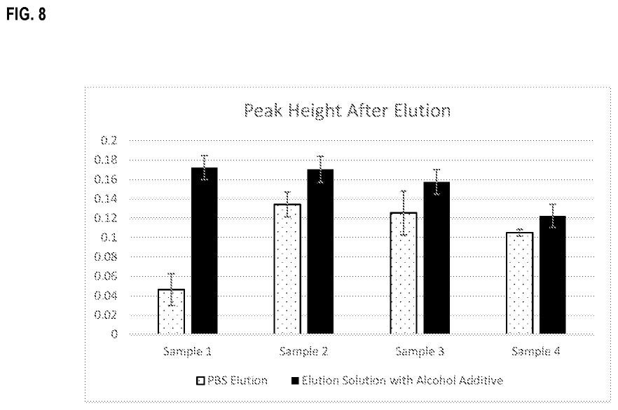

To help with wicking the sample off the swab and fully wet the electrodes on the sensor surface, a hydrophilic membrane can be used to cover the sensor surface. Hydrophilic membranes or meshes can be used to wick liquids onto specified surfaces. As shown in FIG. 7, a 300 mesh woven nylon mesh membrane was adhered to the sensor using a thin double-sided tape cutout. The nylon mesh membrane covered the exposed working, counter, and reference electrodes (FIG. 7). Using the nylon membrane reduced the total volume needed to fully wet all the electrodes from 50-100 μL down to 10-20 μL. Saline solution spiked with the electrochemical biomarker was used to verify this result.

Next, whether adding in some liquid to help dissolve the wax and exudate would be helpful to obtain sufficient liquid for covering all of the electrode surfaces covered with woven nylon mesh membrane was tested. The foam-tipped swab was dipped into a redox spiked saline solution, and the swab was rubbed onto the sensor to see if the solution is wicked by the mesh membrane and covers all electrodes.

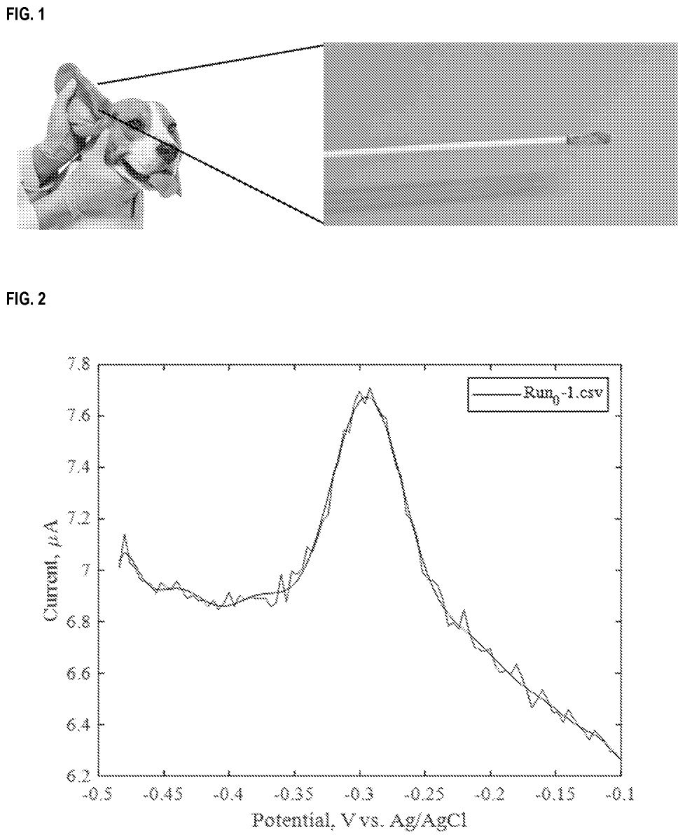

The solution transferred nicely from the foam tipped swab to the membrane and wets the electrodes. To ensure a full coverage, the foam tipped swab was dipped back into the saline solution and reapplied onto the membrane. The electrochemical scan was performed and resulted in peaks consistent with the original baseline pipetting technique, which required 100 μL as seen in FIG. 2.

These results indicate that a combination of using a foam tipped swab, and covering the sensor with a hydrophilic membrane allowed efficient transfer of the liquid for diagnosis.

### Example 10. Extraction of the Exudate from a Foam Swab Using an Alcohol Additive

To improve target molecule recovery, an additive may be mixed with the saline extraction solution, although some additives can interfere with electrochemical measurements. Saline containing different additives was prepared and tested. For example, a phosphate buffered saline (PBS) containing 5% ethanol was prepared by adding to PBS ethanol to 5%. This PBS containing 5% ethanol has the following composition:
137 mM NaCl,
2.7 mM KCl,
10 mM Phosphate buffer.
1 mM MgCl₂
5% ethanol
pH is 7.4

A redox molecule was spiked into cerumen collected from animals. Four separate comparative samples were collected with a foam swab, mixed with 100 μL of PBS containing 5% ethanol, or PBS without ethanol and transferred to a mesh covered electrochemical sensor. The electrochemical measurement was performed and signal compared to unaltered saline solution. As shown in FIG. 8, the cerumen sample extracted with PBS containing 5% ethanol consistently produced a greater signal for of the redox molecule compared to the cerumen sample extracted with PBS alone. These results demonstrated that the extraction solution containing 5% ethanol improved redox molecule recovery.

Similarly, extraction solutions containing other solvent additives, such as methanol, ethanol, isopropyl alcohol, hexane, DMSO, and DMF, ranging in concentration from 1-20% were prepared and tested. Alcohols performed better than other organic solvents tested (data not shown). Ethanol performed better than methanol and isopropyl alcohol (data not shown).

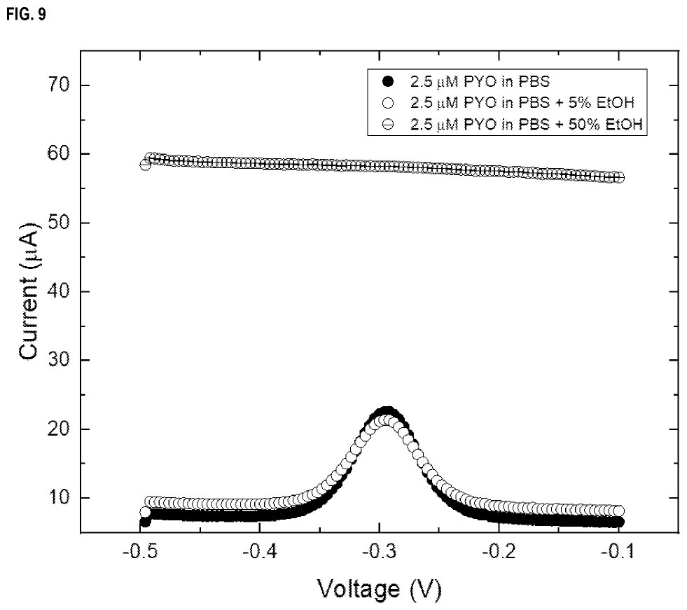

To assay the effect of ethanol on ability of the electrochemical sensor to detent a redox molecule, different amounts of ethanol were added to the PBS containing a 2.5 μM concentration of pyocyanin (PYO), and electrochemical signal was measured. Square-wave voltammetry scans of pyocyanin in different concentrations of ethanol added to phosphate buffered saline solution (PBS) are shown in FIG. 9. The scans in pure PBS and PBS with 5% ethanol showed similar redox peak heights, and were statistically similar when averaged over n=15 samples (FIG. 9). In contrast, as shown in FIG. 9, the scans in PBS containing 50% ethanol showed very high baseline current and a diminished redox peak. These results demonstrate that as the concentration of ethanol in PBS is increased, the redox peak diminishes while the baseline current increases. Thus, extraction solutions containing high levels of the best performing alcohol (ethanol) resulted in diminished electrochemical signal significantly despite better redox molecule extraction (data not shown). Therefore, PBS containing low concentrations of (e.g. from about 0.2% to about 20%, or from about 1% to about 10%, or from about 2.5% to about 7.5%, or about 5%) ethanol efficiently extracts the redox molecules from cerumen, while producing good electrochemical signal.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present technology is not entitled to antedate such publication by virtue of prior invention.

As used herein, all headings are simply for organization and are not intended to limit the disclosure in any manner. The content of any individual section may be equally applicable to all sections.

### EQUIVALENTS

While the invention has been disclosed in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments disclosed specifically herein. Such equivalents are intended to be encompassed in the scope of the following claims.

What is claimed is:

1. A method for detecting an infection in a subject's ear, comprising,
(a) obtaining a sample of cerumen,
the cerumen being adsorbed onto an applicator;
(b) extracting the cerumen from the applicator,
the extraction comprising contacting the applicator with a solvent;
(c) measuring a presence, absence or amount of a compound in the cerumen, optionally wherein the compound is redox-active and associated with the infection,
the measuring comprising contacting the compound with an electrochemical sensor comprising a working electrode and a reference electrode, and
electrochemically measuring a current flow, which is correlated with the presence, absence or amount of the compound.

2. The method of claim 1, further comprising estimating a number of viable cells of a microorganism associated with the infection based on the presence, absence or amount of the compound determined in step (c).

3. The method of claim 1 or claim 2, wherein the cerumen is exuded from the subject's ear.

4. The method of any one of claims 1-3, wherein the cerumen is obtained from the subject's ear canal.

5. The method of any one of claims 1-4, wherein the extracting removes a substantial amount of the cerumen from the applicator.

6. The method of any one of claims 1-5, wherein the extracting removes a substantial amount of the compound from the cerumen.

7. The method of any one of claims 1-6, wherein the extracting occurs in a collection tube.

8. The method of any one of claims 1-7, wherein the extracting occurs on the surface of the electrochemical sensor.

9. The method of any one of claims 1-8, wherein the extracting occurs on a hydrophilic membrane attached to or contacted with the electrochemical sensor.

10. The method of claim 9, wherein the hydrophilic membrane wicks the cerumen from the applicator.

11. The method of claim 9 or claim 10, wherein the hydrophilic membrane sorbs (e.g. absorbs or adsorbs) the solvent.

12. The method of any one of claims 1-11, wherein the applicator is a sterile swab.

13. The method of claim 12, wherein the adsorbent portion of the sterile swab is substantially cotton.

14. The method of claim 12, wherein the adsorbent portion of the sterile swab is substantially foam, substantially calcium alginate, substantially nylon, substantially polyester, substantially polyethylene, substantially flocked polyester, or substantially rayon, optionally wherein the adsorbent portion of the sterile swab is polyurethane foam.

15. The method of any one of claims 1-11, wherein the applicator is a sterile curette.

16. The method of any one of claims 1-15, wherein the infection is a Pseudomonas aeruginosa infection.

17. The method of any one of claims 1-16, wherein the infection is one or more of otitis externa, otitis media, and otitis interna.

18. The method of any one of claims 1-17, wherein the subject is a non-human animal.

19. The method of claim 17, wherein the non-human animal is a captive animal.

20. The method of claim 17, wherein the non-human animal is a pet animal, a farm animal, or a zoo animal.

21. The method of any one of claims 1-17, wherein the subject is a canine.

22. The method of any one of claims 1-21, wherein the solvent is an aqueous solution.

23. The method of claim 17, wherein the aqueous solution comprises ingredients selected from salts, buffering agents, and chelating agents.

24. The method of any one of claims 1-23, wherein the solvent is saline.

25. The method of any one of claims 1-24, wherein the solvent comprises an organic solvent selected from aliphatic hydrocarbons, cyclic hydrocarbons, aromatic hydrocarbons, halogenated hydrocarbons, ketones, amines, esters, alcohols, aldehydes, and ethers.

26. The method of any one of claims 1-21, wherein the solvent comprises ethanol or an aqueous solution thereof.

27. The method of any one of claims 22 to 26, wherein the solvent comprises a phosphate buffered saline (PBS) and an alcohol.

28. The method of claim 27, wherein the alcohol is ethanol.

29. The method of claim 28, wherein the solvent comprises PBS comprising ethanol in an amount of about 0.1% to about 25%, or about 0.2% to about 20%, or about 0.5% to about 15%, or about 1% to about 10%, or about 2% to about 8%, or about 2.5% to about 7.5%, or about 3% to about 7%, or about 4% to about 6%.

30. The method of claim 29, wherein the solvent comprises PBS comprising about 5% ethanol.

31. The method of claim 30, wherein the solvent further comprises about 1 mM MgCl₂.

32. The method of any one of claims 1-31, wherein the solvent further comprises a cerumenolytic.

33. The method of claim 32, wherein the cerumenolytic is selected from dioctyl sodium sulfosuccinate (DOSS), dioctyl calcium sulfosuccinate, urea, sodium bicarbonate, acetic acid, almond oil, peanut oil, rectified camphor oil, olive oil, mineral oil, liquid petrolatum, docusate sodium, triethanolamine polypeptide oleate-condensate, choline salicylate and glycerin.

34. The method of any one of claims 1-33, wherein the compound is a quorum sensing molecule.

35. The method of claim 34, wherein the quorum sensing molecule is a phenazine compound.

36. The method of claim 35, wherein the phenazine compound is pyocyanin.

37. The method of claim 36, wherein the presence of pyocyanin is indicative of the presence or extent of Pseudomonas aeruginosa infection.

38. The method of claim 37, wherein if the current flow through the working electrode is less than about 10 nA, the microorganism associated with the infection is considered nonviable or absent.

39. The method of any one of claims 1-38, wherein the electrochemical measurement is selected from square wave voltammetry, linear sweep voltammetry, staircase voltammetry, cyclic voltammetry, normal pulse voltammetry, differential pulse voltammetry, and chronoamperometry.

40. The method of claim 39, wherein the electrochemical measurement is square wave voltammetry and the current flow is measured in response to one or more square wave potentials.

41. The method of any one of claims 1-40, wherein the electrochemical sensor comprises a second working electrode and the working electrode is one of an oxidizing electrode and a reducing electrode, and the second working electrode is the other of the oxidizing electrode and the reducing electrode.

42. The method of any one of claims 1-41, wherein the presence, absence or amount of the compound is measured as current flow through the working electrode.

43. The method of claim 41 or claim 42, wherein the presence, absence or amount of compound is measured as current flow through the oxidizing electrode and the reducing electrode.

44. The method of any one of claims 1-43, wherein the working electrode is comprised of gold (Au), silver (Ag), platinum (Pt), indium tin oxide (ITO), carbon, carbon nanotubes, carbon nanofibers, graphene, carbon-platinum composites, carbon nanotubes with gold nanoparticles, and any combination thereof.

45. The method of any one of claims 1-44, wherein the reference electrode is comprised of silver (Ag), silver chloride (AgCl), gold (Au), palladium (Pd), and platinum (Pt), and any combination thereof.

46. The method of any one of claims 1-45, wherein the method informs the administration of one or more antibiotics upon a positive test for infection.

47. The method of any one of claims 1-46, wherein the method informs the withholding of one or more antibiotics upon a negative test for infection.

48. The method of any one of claims 1-47, wherein the method informs the selection of an appropriate antibiotic for the infectious agent upon a positive test for infection.

49. A kit for detecting an infection in a subject's ear, comprising,
(a) an applicator suitable for adsorbing a sample of cerumen;
(b) a solvent suitable for extracting the cerumen and/or a compound within the cerumen from the applicator; and, optionally
(c) an electrochemical sensor, the electrochemical sensor comprising a working electrode and a reference electrode and being suitable for electrochemically measuring a current flow through the sensor, which is correlated with the presence, absence or amount of the compound.

50. The kit of claim 49, further comprising one or more of positive control samples, negative control samples, a key for estimating a number of viable cells of a microorganism, and instructions to use.

51. The kit of claim 49 or claim 50, further comprising a collection tube.

52. The kit of any one of claims 49-51, wherein the applicator is a sterile swab or a sterile curette.

53. The kit of claim 52, wherein the adsorbent portion of the sterile swab is made of substantially cotton, substantially foam, substantially calcium alginate, substantially nylon, substantially polyester, substantially polyethylene, substantially flocked polyester, or substantially rayon.

54. The kit of any one of claims 49-53, wherein the solvent is an aqueous solution.

55. The kit of claim 54, wherein the aqueous solution comprises ingredients selected from salts, buffering agents, and chelating agents.

56. The kit of any one of claims 49-55, wherein the solvent is saline.

57. The kit of any one of claims 49-53, wherein the solvent is a non-aqueous solvent or solution.

58. The kit of claim 57, wherein the solvent comprises an organic solvent selected from aliphatic hydrocarbons, cyclic hydrocarbons, aromatic hydrocarbons, halogenated hydrocarbons, ketones, amines, esters, alcohols, aldehydes, and ethers.

59. The kit of claim any one of claim 49-54 or 56-58, wherein the solvent comprises ethanol or an aqueous solution thereof.

60. The kit of any one of claims 54 to 59, wherein the solvent comprises a phosphate buffered saline (PBS) and an alcohol.

61. The kit of claim 60, wherein the alcohol is ethanol.

62. The kit of claim 61, wherein the solvent comprises PBS comprising ethanol in an amount of about 0.1% to about 25%, or about 0.2% to about 20%, or about 0.5% to about 15%, or about 1% to about 10%, or about 2% to about 8%, or about 2.5% to about 7.5%, or about 3% to about 7%, or about 4% to about 6%.

63. The kit of claim 62, wherein the solvent comprises PBS comprising about 5% ethanol.

64. The kit of claim 63, wherein the solvent further comprises about 1 mM MgCl₂.

65. The kit of any one of claims 49-64, wherein the solvent further comprises a cerumenolytic selected from dioctyl sodium sulfosuccinate (DOSS), dioctyl calcium sulfosuccinate, urea, sodium bicarbonate, acetic acid, almond oil, peanut oil, rectified camphor oil, olive oil, mineral oil, liquid petrolatum, docusate sodium, triethanolamine polypeptide oleate-condensate, choline salicylate and glycerin.

66. The kit of claim 50, wherein the positive control comprises Pseudomonas aeruginosa cells or a metabolite thereof.

67. The kit of claim 50, wherein the positive control comprises a quorum sensing molecule.

68. The kit of claim 67, wherein the quorum sensing molecule is a phenazine compound.

69. The kit of claim 68, wherein the phenazine compound is pyocyanin.

70. The kit of any one of claims 49-69, wherein the electrochemical sensor generates a waveform suitable for performing electrochemical measurement selected from square wave voltammetry, linear sweep voltammetry, staircase voltammetry, cyclic voltammetry, normal pulse voltammetry, differential pulse voltammetry, and chronoamperometry.

71. The kit of claim 70, wherein the electrochemical sensor generates a waveform suitable for performing square wave voltammetry, wherein the current flow is measured in response to one or more square wave potentials.

72. The kit of any one of claims 49-71, wherein the electrochemical sensor comprises a second working electrode and the working electrode is one of an oxidizing electrode and a reducing electrode, and the second working electrode is the other of the oxidizing electrode and the reducing electrode.

73. The kit of any one of claims 49-72, wherein the working electrode is comprised of gold (Au), silver (Ag), platinum (Pt), indium tin oxide (ITO), carbon, carbon nanotubes, carbon nanofibers, graphene, carbon-platinum composites, carbon nanotubes with gold nanoparticles, and any combination thereof.

74. The kit of any one of claims 49-73, wherein the reference electrode is comprised of silver (Ag), silver chloride (AgCl), gold (Au), palladium (Pd), and platinum (Pt), and any combination thereof.
